(19) United States
(12) Patent Application Publication
(10) Pub. No.: US 2019/0323968 A1
(43) Pub. Date: Oct. 24, 2019

(54) A FLUID ANALYTICAL DEVICE

(71) Applicant: Orbis Diagnostics Limited, Auckland (NZ)

(72) Inventors: Miriam Cather SIMPSON, Auckland (NZ); David E. WILLIAMS, Auckland (NZ); Michel Karin NIEUWOUDT, Auckland (NZ); Jacob William MARTIN, Cambridge (GB)

(21) Appl. No.: 16/467,486

(22) PCT Filed: Dec. 5, 2017
(86) PCT No.: PCT/US2017/064661

§ 371 Date: Jun. 6, 2019

Related U.S. Application Data
(60) Provisional application No. 62/430,497, filed on Dec. 6, 2016.

(51) Int. Cl. G01N 21/65; G01N 33/04; G01N 21/07; B01L 3/00

(52) U.S. Cl. CPC G01N 21/658; G01N 21/65; G01N 33/04; G01N 2021/056; B01L 3/502715; B01L 3/50273; G01N 21/07

(57) ABSTRACT

A fluid analytical device comprising: a disc (1) rotatable around an axis (2), the disc (1) comprising: a first layer, the first layer comprising: a disc; and at least one microfluidic channel (6) in the disc partially extending from the disc axis (2) to the disc edge; a second layer, the second layer comprising: a disc of substantially the same diameter as the disc of the first layer; and at least one through input port (3) and one through measurement port (4) pair, the input port (3) located near the disc axis (2) and the measurement port (4) located distal from the disc axis (2); wherein when assembled each of the at least one input port (3) and measurement port (4) pair are aligned with one of the at least one microfluidic channels (6); and a disc spinning mechanism (12); a controller (13) for controlling the disc spinning mechanism (12); and a microscope (11) for analysing the fluid through the measurement ports (4) in the rotatable disc (1).

### CROSS-REFERENCE TO RELATED APPLICATIONS

The present application claims priority to, and the benefit of, U.S. Application No. 62/430,497, filed Dec. 6, 2016. The contents of this application is incorporated by reference in its entirety.

### FIELD OF THE INVENTION

The present invention relates to a fluid analytical device.

### BACKGROUND OF THE INVENTION

Raman spectroscopy is a well-accepted analytical detection and identification method for precise measurement of a wide variety of organic, inorganic and biological substances. Combining Raman spectroscopic analysis with microfluidic technology offers the advantage of monitoring samples before, during and after chemical and biochemical processing in a manner that is highly specific, reproducible and automated. Raman spectroscopy offers quantitative analysis of complex samples, particularly with chemometric and machine learning or artificial intelligence applications.

Using microfluidic technology has the potential for automation of quantitative sample analysis in remote, industrial and/or dangerous settings. One impediment encountered in deploying Raman-on-chip technology is the Raman scattering exhibited by the polymeric materials used to make inexpensive chips; the plastics from which chips are constructed often have strong Raman signals. Contamination can be avoided by constructing microfluidic chips from optical quality glasses and by manufacturing optically clear windows of quartz or glass into polymeric chips. However, such approaches are cumbersome and expensive. Further, the introduction of optical windows reduces the Raman signal intensity. One solution is the use of open-surface microfluidic channels. However, filling is too slow for many applications and air bubble-entrapment can be a significant issue.

Various other references to the prior art and its associated problems are made throughout the following description.

Each object is to be read disjunctively with the object of at least providing the public with a useful choice.

The present invention aims to overcome, or at least alleviate, some or all of the aforementioned problems.

### SUMMARY OF THE INVENTION

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning—i.e. they will be taken to mean an inclusion of the listed components which the use directly references, and possibly also of other non-specified components or elements.

According to one aspect, the present invention provides a fluid analytical device, comprising:

a disc rotatable around an axis, the disc comprising:

a first layer, the first layer comprising:
a disc; and
at least one microfluidic channel in the disc partially extending from the disc axis to the disc edge;

a second layer, the second layer comprising:
a disc of substantially the same diameter as the disc of the first layer; and
at least one through input port and one through measurement port pair, the input port located near the disc axis and the measurement port located distal from the disc axis;

wherein when assembled each of the at least one input port and measurement port pair are aligned with one of the at least one microfluidic channels; and
a disc spinning mechanism;
a controller for controlling the disc spinning mechanism; and
a measurement system for analysing the fluid through the measurement ports in the rotatable disc.

Preferably the first and second layers are bonded together.

Preferably the first and second layers are bonded together with an adhesive that does not react to or dissolve in the fluid to be analysed.

Preferably the microfluidic channel in the first layer is tapered towards the base of the channel.

Preferably the taper is such that the angle formed by the walls of the channel at the base of the channel is less than 60 degrees and greater than 10 degrees.

Preferably the angle formed by the walls of the channel at the base of the channel is less than 0.5×(90−θ) degrees, where θ denotes the contact angle formed by the fluid with the material forming the channel walls, at the air-fluid-material interface.

Preferably wherein the discs are formed of a material that does not dissolve in or react to the fluid to be analysed.

Preferably the controller is operable to rotate the disc with controlled angular acceleration between an initial rotation rate and a final rotation rate.

Preferably the initial rotation rate is zero and the final rotation rate is 3000 rpm.

Preferably the final rotation rate in units of radian/second does not exceed a value given by the formula

$${ω}_{max} = \sqrt{\frac{2 γ sin ( θ )}{d ρ {r}_{i} {r}_{o}}}$$

where γ denotes the interfacial tension at the fluid-air interface, θ denotes the contact angle formed by the fluid with air at the interface with the first disc material, where it is exposed to air by the measurement port, d denotes the width of the open channel exposed by the measurement port, measured across the channel at its opening to the air, ρ denotes the density of the fluid, rᵢ denotes the radial distance from the centre of the disc of the start of the open segment of the channel and rₒ denotes the radial distance from the centre of the disc of the end of the open segment of the channel.

Preferably the initial rotation rate in units of radian/second is greater than a value given by the formula

$${ω}_{min} = {ω}_{max} \sqrt{\frac{{r}_{o}}{{r}_{i}} cotangent ( θ )}$$

and is less than the final rotation rate.

Preferably the angular acceleration is controlled in the range 2 radian/s² to 200 radian/s².

Preferably the controller is operable to rotate the disc at a minimum speed to overcome the surface tension at the edges of the measurement port.

Preferably the channels in the first layer are a maximum width of 250 μm and a depth of approximately 1 mm.

Preferably the size of the input ports is less than or equal to the width of the channel.

Preferably the size of the measurement ports is greater than the channel width.

Preferably the second layer further includes an outlet port.

Preferably the first layer further includes a catchment chamber distal from the disc axis and further from the disc axis than the measurement port.

Preferably the first layer further includes a catchment chamber distal from the disc axis and further from the disc axis than the measurement port and wherein the second layer further includes an outlet port distal from the disc axis and further from the disc axis than the catchment chamber.

Preferably the microfluidic channels are aligned along a radial line.

Preferably the measurement system detects scattered or emitted light.

Preferably the measurement system includes a reader head that is fibre based and is used to deliver light and to collect the scattered or emitted light.

Alternatively, the measurement system is a Raman microscope.

Alternatively, the measurement system is a system that measures scattering and emission of light in response to one or more incident beams of light in the wavelength range 1.2 μm to 250 nm.

Alternatively, the measurement system is a system that measures scattering of light in response to one or more incident beams of light in the wavelength range 1.2 μm to 250 nm.

Alternatively, the measurement system is a system that measures elastic scattering of light in response to one or more incident beams of light in the wavelength range 1.2 μm to 250 nm.

Alternatively, the measurement system is a system that measures inelastic scattering of light in response to one or more incident beams of light in the wavelength range 1.2 μm to 250 nm.

Alternatively, the measurement system is a system that measures inelastic scattering of light that is spontaneous vibrational Raman scattering in response to one or more incident beams of light in the wavelength range 1.2 μm to 250 nm.

Alternatively, the measurement system is a system that measures Raman scattering that is resonantly enhanced in response to one or more incident beams of light in the wavelength range 1.2 μm to 250 nm.

Alternatively, the measurement system is a system that measures Raman scattering that is surface enhanced in response to one or more incident beams of light in the wavelength range 1.2 μm to 250 nm.

Alternatively, the measurement system is a system that measures using Raman scattering in response to one or more incident beams of light, that reflects a higher-order interaction of the light with the fluid, including stimulated Raman scattering and coherent Anti-Stokes Raman scattering.

Preferably the one or more incident beams are pulsed.

Alternatively, the one or more incident beams are a continuous wave.

Alternatively, the one or more incident beams are narrow band and less than or equal to 10 nm in spectral width.

Alternatively, the one or more incident beams are broad band and more than 10 nm in spectral width.

Alternatively, the one or more incident beams contain a single continuous wavelength range.

Alternatively, the one or more incident beams are structured to contain two or more continuous wavelength ranges.

Preferably the device is for the spectroscopic analysis of milk.

### BRIEF DESCRIPTION OF THE DRAWINGS

Embodiments of the present invention will now be described, by way of example only, with reference to the accompanying drawings.

FIG. 1 shows an embodiment of a disc of the present invention.

FIG. 2A shows the first layer or a disk of the present invention.

FIG. 2B shows the second layer or a disk of the present invention.

FIG. 3 shows a further embodiment of a disc of the present invention.

FIG. 4 shows a block diagram of the analytical device of the present invention.

FIG. 5 shows a block diagram of the microfluidic channel in a further embodiment of the present invention.

FIG. 6 shows a block diagram of the microfluidic channel in a further embodiment of the present invention.

FIG. 7 shows a block diagram of the microfluidic channel in a further embodiment of the present invention.

### DETAILED DESCRIPTION OF THE INVENTION

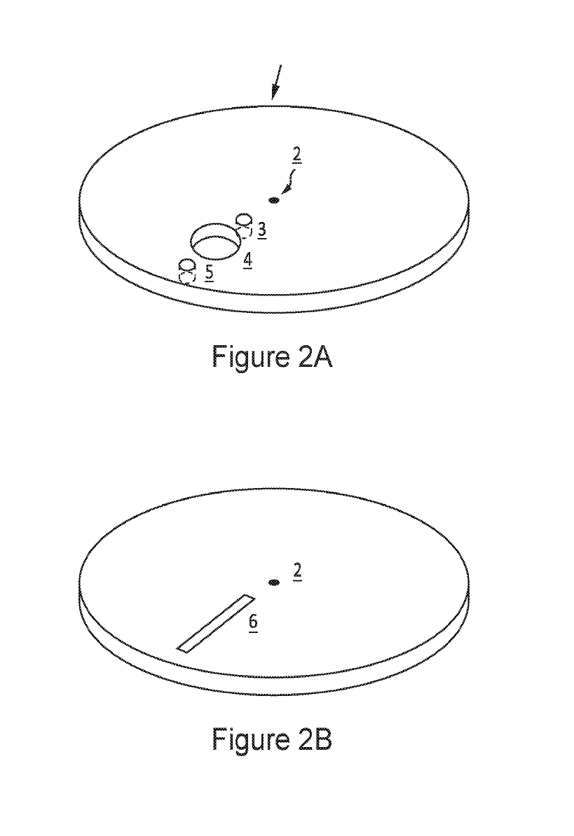

Referring to the Figures the analytical device of the present invention will be described. A disc 1, is rotatable around an axis 2 by a motor or disc spinning mechanism 12. The motor 12 is controlled by a controller 13. The disc is comprised of two layers seen in FIGS. 2A and 2B.

The second layer seen in FIG. 2A includes a fluid inlet port 3 a measurement port 4 from which analytical measurements may be taken. The size of the measurement ports is greater than the channel width. The second layer further includes an outlet port 5. The inlet 3, measurement 4, and outlet 5 ports extend through the thickness of the first layer. While the inlet 3, measurement 4, and outlet 5 ports are illustrated as round any suitable shape may be used such as oval or rectangular.

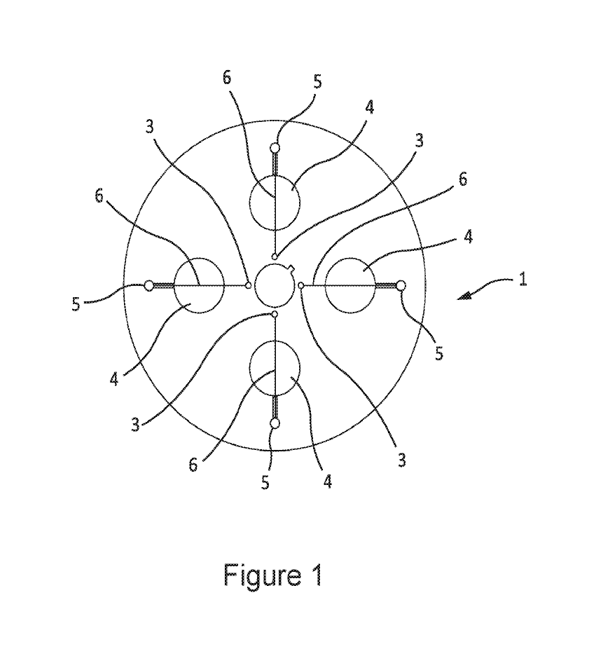

The first layer seen in FIG. 2B includes a microfluidic channel 6 cut into the layer. When the first and second layers are assembled and bonded for example with an adhesive the channel 6 lines up with the inlet 3, measurement 4, and outlet 5 ports. The alignment is in one embodiment along a radial line. In FIG. 1 a disk is shown with multiple channels 6 aligned with multiple inlet 3, measurement 4, and outlet 5 ports.

Discs layers are in one embodiment formed of a material that does not dissolve in or react to the fluid to be analysed.

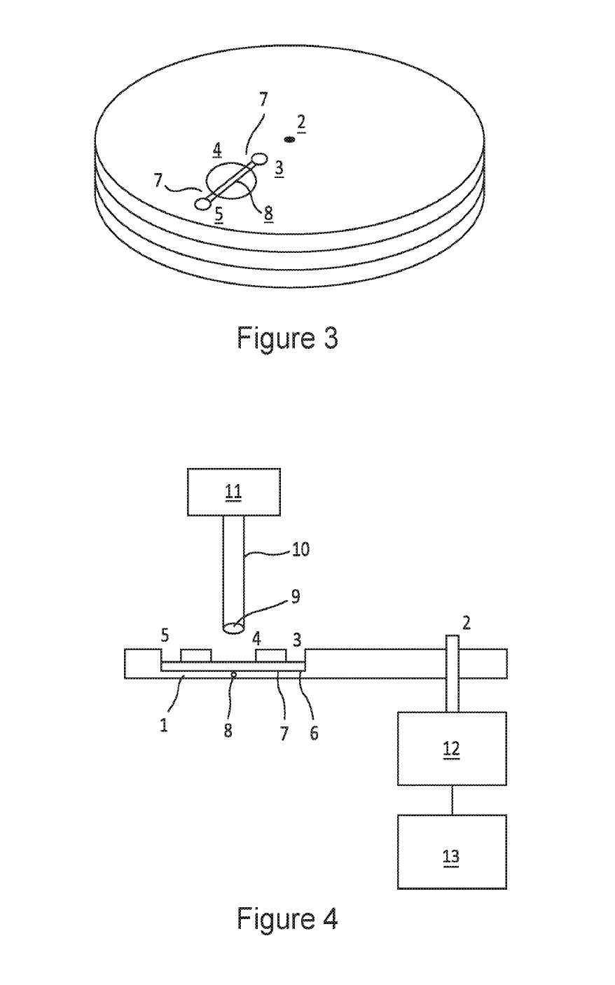

Referring to FIG. 3 the analytical device further includes a lens 9 aligned with the measurement port 4. The lens 9 is connected via an optical fibre 10 to a measurement system or microscope 11. The reader head of the measurement system is fibre based and is used to deliver the light and collect the scattered or emitted light. In one embodiment the measurement device 11 is a Raman spectrometer. The open part 8 of the channel 6 below the measurement port 4 allows the measurement device 11 to measure fluid in the channel 6.

The bonding of the first and second layers is in one embodiment with an adhesive that does not react to or dissolve in the fluid to be analysed.

The microfluidic channel 6 in the first layer is tapered towards the base of the channel and the taper is such that the angle formed by the walls of the channel at the base of the microfluidic channel 6 is less than 60 degrees and greater than 10 degrees. The angle formed by the walls of the microfluidic channel 6 at the base of the microfluidic channel is less than 0.5×(90−θ) degrees, where θ denotes the contact angle formed by the fluid with the material forming the channel walls, at the air-fluid-material interface.

Controller 13 is operable to rotate the disc 1 using motor 12 with controlled angular acceleration between an initial rotation rate and a final rotation rate. In the preferred embodiment the initial rotation rate is zero and the final rotation rate is 3000 rpm. Other final rotation speeds may be used. The maximum value of the final rotation speed is calculated using the formula

$${ω}_{max} = \sqrt{\frac{2 γ sin ( θ )}{d ρ {r}_{i} {r}_{o}}}$$

where γ denotes the interfacial tension at the fluid-air interface, θ denotes the contact angle formed by the fluid with air at the interface with the first disc material, where it is exposed to air by the measurement port, d denotes the width of the open channel exposed by the measurement port, measured across the channel at its opening to the air, ρ denotes the density of the fluid, rᵢ, denotes the radial distance from the centre of the disc of the start of the open segment of the channel and rₒ denotes the radial distance from the centre of the disc of the end of the open segment of the channel.

Controller 13 may be any suitable electronic computing device including a single chip device.

Further the minimum initial rotation rate (in units of radian/second) is calculated using the formula

$${ω}_{min} = {ω}_{max} \sqrt{\frac{{r}_{o}}{{r}_{i}} cotangent ( θ )}$$

In the preferred embodiment angular acceleration is controlled in the range 2 radian/s² to 200 radian/s²

The controller rotates the disc at a minimum speed to overcome the surface tension at the edges of the measurement port depending on the fluid to be analysed and at a maximum rotation speed such that the fluid to be analysed is retained in the part of the channel that is open to air and does not overflow.

In one embodiment channels 6 in the first layer are a maximum width of 250 μm and a depth of approximately 1 mm.

The fluid analytical device is in one embodiment for the spectroscopic analysis of milk but may be used for analysis of other fluids.

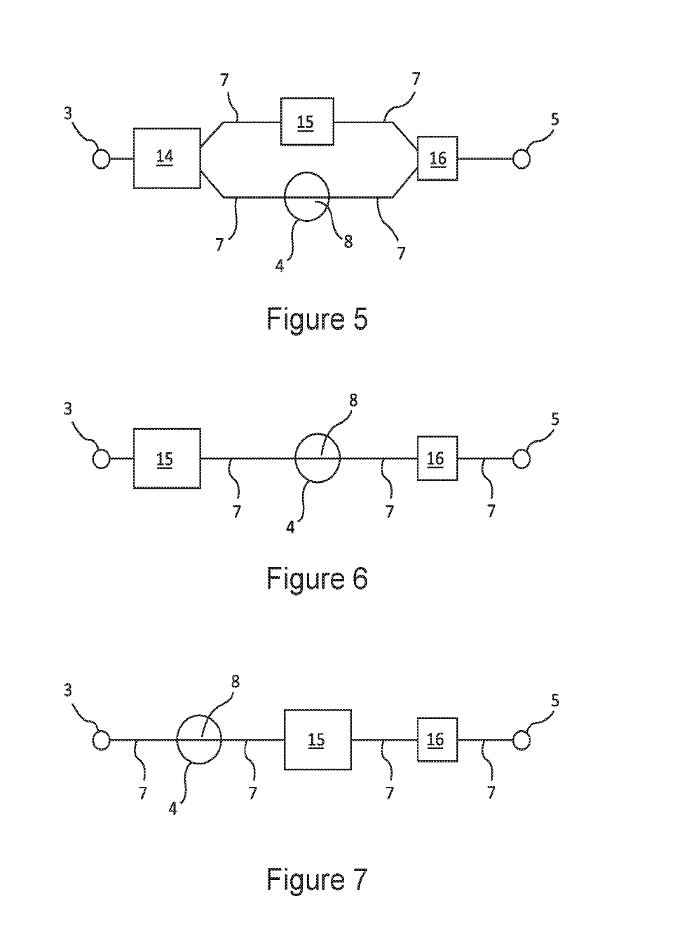

In a further embodiment illustrated in FIG. 5 the microfluidic channel 6 may be split using an aliquoting and flow divider 14 and provide a port 15 for measurements other than the port 4 for Raman spectroscopy. Optionally a collection port 16 may also be provided.

In yet a further embodiment illustrated in FIGS. 6 and 7 the microfluidic channel 6 may provide a port 15 for measurements other than the port 4 for Raman spectroscopy 8. The port 15 may be either before (illustrated in FIG. 6) or after (illustrated in FIG. 7) the port 4 for Raman spectroscopy. Optionally a collection port 16 may also be provided.

While two options for further measurements have been illustrated it is to be understood other combinations of flow dividers and one or more other ports may be provided.

An open-channel in a disc that spins is useful for a spectroscopic detection system in which:

(1) Light from the near-IR to the ultraviolet (1.2 μm to 250 nm) is incident on the sample in the open channel through the un-covered interface;

(2) The sample in the open channel response is in the form of light that is scattered or emitted, some of which leaves the fluid sample through the un-covered interface; and

(3) That light that is scattered and/or emitted by the sample through the un-covered interface is collected, detected and analysed by a detection system.

The advantage of the open channel is to improve the accuracy, sensitivity and precision of the photonic detection and analysis system, by removing the material from which the fluidic device is constructed so that it cannot impede the passage of light, and/or change the spectral character of the light by either subtracting or adding spectral signatures to the light, and/or change the noise characteristics of the light; where “the light” refers to the light traveling in both directions across the channel interface; that is. “The light” refers to photons that are incident on the sample inside the channel, and to “the light” that is scattered and/or emitted by the sample inside the channel in the fluidic device.

The spectroscopic detection system using the open-channel in a disc that spins may be used for measurement systems that measure:

(1) Scattering and emission of light in response to one or more incident/incident excitation beams of light in the wavelength range 1.2 μm to 250 nm;

(2) Scattering of light in response to one or more incident/incident excitation beams of light in the wavelength range 1.2 μm to 250 nm;

(3) Elastic scattering of light in response to one or more incident/incident excitation beams of light in the wavelength range 1.2 μm to 250 nm;

(4) Inelastic scattering of light in response to one or more incident/incident excitation beams of light in the wavelength range 1.2 μm to 250 nm;

(5) Inelastic scattering of light that is spontaneous vibrational Raman scattering in response to one or more incident/incident excitation beams of light in the wavelength range 1.2 μm to 250 nm;

(6) Raman scattering that is resonantly enhanced in response to one or more incident/incident excitation beams of light in the wavelength range 1.2 μm to 250 nm;

(7) Raman scattering that is surface enhanced (Surface Enhanced Raman Scattering) in response to one or more incident/incident excitation beams of light in the wavelength range 1.2 μm to 250 nm;

(8) Raman scattering in response to one or more incident/incident excitation beam that reflects a higher-order interaction of the light with the fluid, including Stimulated Raman Scattering and Coherent Anti-Stokes Raman Scattering.

For each of the measurement systems (1-8 above) the one or more incident/incident excitation beams may be varied, such that:

the one or more incident/incident excitation beams are pulsed;

the one or more incident/incident excitation beams are a continuous wave;

the one or more incident/incident excitation beams are narrow band (less than or equal to 10 nm in spectral width);

the one or more incident/incident excitation beams are broad band (more than 10 nm in spectral width);

the one or more incident/incident excitation beams contain a single continuous wavelength range; and

the one or more incident/incident excitation r beams are structured to contain two or more continuous wavelength ranges.

While the present invention has been illustrated by the description of the embodiments thereof, and while the embodiments have been described in detail, it is not the intention of the Applicant to restrict or in any way limit the scope of the appended claims to such detail. Further, the above embodiments may be implemented individually, or may be combined where compatible. Additional advantages and modifications, including combinations of the above embodiments, will readily appear to those skilled in the art. Therefore, the invention in its broader aspects is not limited to the specific details, representative apparatus and methods, and illustrative examples shown and described. Accordingly, departures may be made from such details without departure from the spirit or scope of the Applicant's general inventive concept.

1. A fluid analytical device, comprising:
a disc rotatable around an axis, the disc comprising:
a first layer, the first layer comprising:
a disc; and
at least one microfluidic channel in the disc partially extending from the disc axis to the disc edge;
and
a second layer, the second layer comprising:
a disc of substantially the same diameter as the disc of the first layer; and
at least one through input port and one through measurement port pair, the input port located near the disc axis and the measurement port located distal from the disc axis;
wherein when assembled, each of the at least one input port and measurement port pair are aligned with one of the at least one microfluidic channels;
a disc spinning mechanism;
a controller for controlling the disc spinning mechanism;
and
a measurement system for analysing the fluid through the measurement ports in the rotatable disc.

2. The fluid analytical device as claimed in claim 1, wherein:
the first and second layers are bonded together,
the first and second layers are bonded together with an adhesive that does not react to or dissolve in the fluid to be analysed,
the microfluidic channel in the first layer is tapered towards the base of the channel.
the discs are formed of a material that does not dissolve in or react to the fluid to be analysed,
the controller is operable to rotate the disc with controlled angular acceleration between an initial rotation rate and a final rotation rate,
the controller is operable to rotate the disc at a minimum speed to overcome the surface tension at the edges of the measurement port,
the channels in the first layer are a maximum width of 250 μm and a depth of approximately 1 mm,
the size of the input ports is less than or equal to the width of the channel,
the size of the measurement ports is greater than the channel width,
the second layer further includes an outlet port,
the first layer further includes a catchment chamber distal from the disc axis and further from the disc axis than the measurement port,
the first layer further includes a catchment chamber distal from the disc axis and further from the disc axis than the measurement port and wherein the second layer further includes an outlet port distal from the disc axis and further from the disc axis than the catchment chamber,
the microfluidic channels are aligned along a radial line,
the measurement system detects scattered or emitted light,
the measurement system includes a reader head that is fibre based and is used to deliver light and to collect the scattered or emitted light,
the measurement system is a Raman microscope,
the measurement system is a system that measures scattering and emission of light in response to one or more incident beams of light in the wavelength range 1.2 μm to 250 nm,
the measurement system is a system that measures scattering of light in response to one or more incident beams of light in the wavelength range 1.2 μm to 250 nm,
the measurement system is a system that measures elastic scattering of light in response to one or more incident beams of light in the wavelength range 1.2 μm to 250 nm,
the measurement system is a system that measures inelastic scattering of light in response to one or more incident beams of light in the wavelength range 1.2 μm to 250 nm,
the measurement system is a system that measures inelastic scattering of light that is spontaneous vibrational Raman scattering in response to one or more incident beams of light in the wavelength range 1.2 μm to 250 nm,
the measurement system is a system that measures Raman scattering that is resonantly enhanced in response to one or more incident beams of light in the wavelength range 1.2 μm to 250 nm,
the measurement system is a system that measures Raman scattering that is surface enhanced in response to one or more incident beams of light in the wavelength range 1.2 μm to 250 nm,
the measurement system is a system that measures using Raman scattering in response to one or more incident beams of light, that reflects a higher-order interaction of the light with the fluid, including stimulated Raman scattering and coherent Anti-Stokes Raman scattering,
or
the device is for the spectroscopic analysis of milk.

3.-4. (canceled)

5. The fluid analytical device as claimed in claim 2, wherein the taper is such that the angle formed by the walls of the channel at the base of the channel is less than 60 degrees and greater than 10 degrees.

6. The fluid analytical device as claimed in claim 5, wherein the angle formed by the walls of the channel at the base of the channel is less than 0.5×(90−θ) degrees, where θ denotes the contact angle formed by the fluid with the material forming the channel walls, at the air-fluid-material interface.

7-8. (canceled)

9. The fluid analytical device as claimed in claim 2, wherein:
the initial rotation rate is zero and the final rotation rate is 3000 rpm,
the final rotation rate in units of radian/second does not exceed a value given by the formula a ωₘₐₓ=\sqrt{2γ sin(θ)/dρrᵢrₒ}, where γ denotes the interfacial tension at the fluid-air interface, θ denotes the contact angle formed by the fluid with air at the interface with the first disc material, where it is exposed to air by the measurement port, d denotes the width of the open channel exposed by the measurement port, measured across the channel at its opening to the air, ρ denotes the density of the fluid, n denotes the radial distance from the centre of the disc of the start of the open segment of the channel and rₒ denotes the radial distance from the centre of the disc of the end of the open segment of the channel,
initial rotation rate in units of radian/second is greater than a value given by given by the formula ωₘᵢₙ=ωₘₐₓ\sqrt{rₒ/rᵢ cotangent(θ)} and is less than the final rotation rate,
or
the angular acceleration is controlled in the range 2 radian/s² to 200 radian/s².

10-31. (canceled)

32. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

33-37. (canceled)

38. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

39-43. (canceled)

44. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

45-49. (canceled)

50. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

51-55. (canceled)

56. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

57-61. (canceled)

62. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

63-67. (canceled)

68. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

69-73. (canceled)

74. The fluid analytical device as claimed in claim 2, wherein:
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

75-80. (canceled)

81. A fluid analytical device, comprising:
a disc rotatable around an axis, the disc comprising:
a first layer, the first layer comprising:
a disc; and
at least one microfluidic channel in the disc partially extending from the disc axis to the disc edge;
a second layer, the second layer comprising:
a disc of substantially the same diameter as the disc of the first layer; and
at least one through input port and one through measurement port pair, the input port located near the disc axis and the measurement port located distal from the disc axis;
wherein when assembled each of the at least one input port and measurement port pair are aligned with one of the at least one microfluidic channels;
and
a disc spinning mechanism;
a controller for controlling the disc spinning mechanism;
and
a measurement system for analysing the fluid through the measurement ports in the rotatable disc.

82. The fluid analytical device as claimed in claim 81, wherein:
the first and second layers are bonded together,
the microfluidic channel in the first layer is tapered towards the base of the channel,
the discs are formed of a material that does not dissolve in or react to the fluid to be analysed.
the controller is operable to rotate the disc with controlled angular acceleration between an initial rotation rate and a final rotation rate,
the controller is operable to rotate the disc at a minimum speed to overcome the surface tension at the edges of the measurement port,
the channels in the first layer are a maximum width of 250 μm and a depth of approximately 1 mm,
the size of the input ports is less than or equal to the width of the channel,
the size of the measurement ports is greater than the channel width,
the second layer further includes an outlet port,
the first layer further includes a catchment chamber distal from the disc axis and further from the disc axis than the measurement port,
the first layer further includes a catchment chamber distal from the disc axis and further from the disc axis than the measurement port and wherein the second layer further includes an outlet port distal from the disc axis and further from the disc axis than the catchment chamber,
the microfluidic channels are aligned along a radial line,
the measurement system detects scattered or emitted light,
the measurement system includes a reader head that is fibre based and is used to deliver light and to collect the scattered or emitted light,
the measurement system is a Raman microscope,
the measurement system is a system that measures scattering and emission of light in response to one or more incident beams of light in the wavelength range 1.2 μm to 250 nm,
the measurement system is a system that measures scattering of light in response to one or more incident beams of light in the wavelength range 1.2 μm to 250 nm,
the measurement system is a system that measures elastic scattering of light in response to one or more incident beams of light in the wavelength range 1.2 μmη to 250 nm,
the measurement system is a system that measures inelastic scattering of light in response to one or more incident beams of light in the wavelength range 1.2 μm to 250 nm,
the measurement system is a system that measures inelastic scattering of light that is spontaneous vibrational Raman scattering in response to one or more incident beams of light in the wavelength range 1.2 μm to 250 nm,
the measurement system is a system that measures Raman scattering that is resonantly enhanced in response to one or more incident beams of light in the wavelength range 1.2μη to 250 nm,
the measurement system is a system that measures Raman scattering that is surface enhanced in response to one or more incident beams of light in the wavelength range 1.2μη to 250 nm,
the measurement system is a system that measures using Raman scattering in response to one or more incident beams of light, that reflects a higher-order interaction of the light with the fluid, including stimulated Raman scattering and coherent Anti-Stokes Raman scattering,
or
the device is for the spectroscopic analysis of milk.

83. The fluid analytical device as claimed in claim 82, wherein:
the first and second layers are bonded together with an adhesive that does not react to or dissolve in the fluid to be analysed,
the taper is such that the angle formed by the walls of the channel at the base of the channel is less than 60 degrees and greater than 10 degrees,
the initial rotation rate is zero and the final rotation rate is 3000 rpm,
the initial rotation rate in units of radian/second is greater than a value given by given by the formula u>min=(max\hat{ }cotangent(6>) and is less than the final rotation rate,
the angular acceleration is controlled in the range 2 radian/s2 to 200 radian/s2,
the one or more incident beams are pulsed,
the one or more incident beams are a continuous wave,
the one or more incident beams are narrow band and less than or equal to 10 nm in spectral width,
the one or more incident beams are broad band and more than 10 nm in spectral width,
the one or more incident beams contain a single continuous wavelength range,
or
the one or more incident beams are structured to contain two or more continuous wavelength ranges.

84-85. (canceled)

86. The fluid analytical device as claimed in claim 83, wherein the angle formed by the walls of the channel at the base of the channel is less than 0.5χ(90−Θ) degrees, where Θ denotes the contact angle formed by the fluid with the material forming the channel walls, at the air-fluid-material interface.

87-89. (canceled)

90. The fluid analytical device as claimed in claim 82, wherein the final rotation rate in units of radian/second does not exceed a value given by the formula
$${ω}_{max} = \sqrt{\frac{2 γ sin ( θ )}{d ρ {r}_{i} {r}_{o}}}$$
where γ denotes the interfacial tension at the fluid-air interface, θ denotes the contact angle formed by the fluid with air at the interface with the first disc material, where it is exposed to air by the measurement port, d denotes the width of the open channel exposed by the measurement port, measured across the channel at its opening to the air, ρ denotes the density of the fluid, rᵢ denotes the radial distance from the centre of the disc of the start of the open segment of the channel and rₒ denotes the radial distance from the centre of the disc of the end of the open segment of the channel.

91. The fluid analytical device as claimed in claim 82, wherein the initial rotation rate in units of radian/second is greater than a value given by given by the formula
$${ω}_{min} = {ω}_{max} \sqrt{\frac{{r}_{o}}{{r}_{i}} cotangent ( θ )}$$
and is less than the final rotation rate.

92-118. (canceled)
